(19) United States
(12) Patent Application Publication
(10) Pub. No.: US 2024/0172977 A1
(43) Pub. Date: May 30, 2024

(54) AUTOMATIC DETECTION SYSTEM FOR DEPRESSIVE DISORDER BASED ON HIGH FREQUENCY AUDITORY STEADY-STATE RESPONSE

(71) Applicant: TIANJIN UNIVERSITY, Tianjin (CN)

(72) Inventors: Xiaoya LIU, Tianjin (CN); Shuang LIU, Tianjin (CN); Dong MING, Tianjin (CN); Wenquan ZHANG, Tianjin (CN); Yufeng KE, Tianjin (CN); Jie LI, Tianjin (CN); Long CHEN, Tianjin (CN)

(21) Appl. No.: 18/102,495

(22) Filed: Jan. 27, 2023

(30) Foreign Application Priority Data
Nov. 17, 2022 (CN) 202211441030.7

(51) Int. Cl. A61B 5/16; A61B 5/00

(52) U.S. Cl. CPC A61B 5/165; A61B 5/7264

(57) ABSTRACT

The present disclosure discloses an automatic detection system for depressive disorder based on high frequency ASSR. The system includes an auditory stimulation module, a data acquisition module, a signal processing module, a depression detection module and an output module; the auditory stimulation module presents 40 Hz frequency-increasing sound stimulation signals to a user; the data acquisition module acquires EEG signals by a non-intrusive method for preprocessing to obtain ASSR data; the signal processing module extracts depressive disorder-related EEG features from the ASSR data; the depression detection module identifies a user depression state through decision fusion of the depressive disorder-related EEG features; and the output module identifies the user depression state according to the EEG features to generate an evaluation report for abnormity in EEG response, and feeds it back to the user.

### CROSS-REFERENCE TO RELATED APPLICATION

This application claims priority from the Chinese patent application 202211441030.7 filed Nov. 17, 2022, the content of which is incorporated herein in the entirety by reference.

### TECHNICAL FIELD

The present disclosure belongs to disease surveillance equipment, and particularly relates to an automatic detection system for depressive disorder based on high frequency auditory steady-state response.

### BACKGROUND ART

Depressive disorder is a highly prevalent mental disease characterized by significant and persistent low mood. Meanwhile, it is the most important type of mental diseases in modern people. According to incomplete statistics from the World Health Organization, depressive disorder will be the leading disabling disease by 2030. In clinically, the diagnosis of depressive disorder remains entirely based on subjective symptomatology. However, these subjective assessments have certain inconsistency and concealment, which necessitates the identification of objective and reliable biomarkers to improve diagnosis and treatment of depressive disorder. Recently, accumulating studies have confirmed that patients with depressive disorder show abnormal gamma oscillations. It is revealed that gamma oscillations are derived from a synaptic interaction between parvalbumin (PV) inhibitory gamma-aminobutyric acid (GABA)-ergic interneurons and excitatory pyramidal neurons. However, gamma oscillations are typically disregarded due to their low amplitude qualities and possible physiological artifacts. One prevalent approach to enhance gamma activity is through entrainment by exogenous rhythmic stimulation. Wherein, auditory steady-state response (ASSR) is a cortical oscillation entrained to both the frequency and phase of periodic auditory stimuli, generated by a whole auditory nervous system, and has a good frequency specificity. Gamma ASSR has been proposed as an effective probe to evaluate gamma synchronous activity. In addition, numerous previous studies have confirmed that the 40 Hz ASSR is impaired in patients with depressive disorder, especially in power, phase coherence, functional connectivity and other aspects.

Therefore, the present disclosure provides an automatic detection system for depressive disorder based on high frequency auditory steady-state response. The gamma ASSR features of a user are acquired by performing the extraction of ERSP, ITC, functional connectivity and other related features on preprocessed acquired cerebral cortex ASSR data through a frequency-increasing chirp ASSR paradigm. Automatic detection and comprehensive evaluation of user depression are conducted through decision fusion to facilitate the early identification of clinical depressive disorder and provide auxiliary support for subsequent clinical diagnosis of depression.

### SUMMARY

In order to solve the problems in the prior art, the present disclosure is intended to provide an automatic detection system for depressive disorder based on high frequency auditory steady-state response; the system achieves stable induction to gamma ASSR by frequency-increasing chirp signals designed in an auditory stimulation module to enhance ASSR response. Cerebral cortex response signals are recorded and preprocessed by a data acquisition module while an auditory module presents stimulation, and the preprocessed ASSR data is transmitted to a signal processing module to extract ASSR features closely related to the depressive disorder, namely multidimensional feature parameters such as ERSP, ITC, and WPLI; then, these feature parameters serve as inputs of a depression detection module to identify a user through decision fusion; and identification results are transmitted to an output module, by which the identification results are fed back to the user, and therefore the automatic detection and output of the user depression are achieved. Through design of frequency-increasing chirp signals based on the cochlear traveling wave delay, stable induction of the gamma ASSR is enhanced and the signal-to-noise ratio of gamma ASSR is increased. A mapping relationship between the depressive disorder and gamma ASSR is decoded based on a previously constructed electroencephalogram database of normal people versus patients with depressive disorder, and depressive disorder-related ASSR features are obtained. The comprehensive evaluation of depression state for the user is achieved through extraction of multidimensional feature parameters such as ERSP, ITC, WPLI from the user ASSR signals during auditory stimulation, and the automatic detection and output of the user depression are achieved through decision fusion. The present disclosure may achieve early identification of the depressive disorder to assist the user in receiving timely treatment, which may be extended to neuropsychology, clinical medicine and other fields to yield considerable social and economic benefits. In the automatic detection system for the depressive disorder based on the high frequency auditory steady-state response designed by the present disclosure, a frequency-increasing chirp auditory stimulation paradigm capable of stably inducing the gamma ASSR is introduced innovatively, a feasible reference is provided for improvement of ASSR response intensity, and a new method and a new thought are provided for exploration of auditory perception disorders arising from other mental diseases. In addition, an effective approach may be provided for detection of the depressive disorder in conjunction with multidimensional feature parameters of the ASSR induced by the paradigm, and it is expected to provide auxiliary support for precise diagnosis in clinic.

The solution to the practical problem of the present disclosure is achieved by adopting the following technical solution:

An automatic detection system for depressive disorder based on high frequency auditory steady-state response (ASSR) includes an auditory stimulation module, a data acquisition module, a signal processing module, a depression detection module and an output module, wherein

the auditory stimulation module inputs 40 Hz frequency-increasing sound stimulation signals to a user to obtain EEG signals;

the data acquisition module acquires the EEG signals by a non-intrusive method, and performs preprocessing to obtain ASSR data;

the signal processing module extracts depressive disorder-related EEG features from the ASSR data, wherein:

the depressive disorder-related EEG features include an event-related spectral perturbation (ERSP) feature, an inter-trial phase coherence (ITC) feature and a weighted phase-lag-index (WPLI) feature;

the ERSP feature is used for measuring power change of 40 Hz ASSR;

the ITC feature is used for measuring an ITC value during auditory stimulation;

the WPLI feature representing the connectivity between the brain regions is used for measuring a change in the connectivity between the brain regions during auditory stimulation;

the depression detection module identifies a user depression state through decision fusion of the depressive disorder-related EEG features; and

the output module identifies the user depression state according to the EEG features to generate an evaluation report for abnormality in EEG response, and feeds it back to the user.

Furthermore, the signal processing module extracts the ERSP feature, which includes the following steps:

performing frequency domain feature analysis through short-time Fourier transform (STFT) by the signal processing module, extracting the ASSR data induced by 40 Hz chirp stimulation under montage of frontal lobes and temporal lobes, and calculating a power value of each trial according to the following formula:

$$ERSP ( f , t ) = \frac{1}{m} {\sum }_{k = 1}^{m} ( {{F}_{k} ( f , t )}^{2} ) \qquad ( 1 )$$

where, f represents frequency, t represents time, k is a mark number of a corresponding trial, m is a total number of n groups of trials, Fₖ(f, t)² represents a corresponding power value at a frequency of f and the moment of t in a kth trial;

performing background interference reduction and individual baseline difference on the ASSR data by the signal processing module through the following formula:

$${{F}_{k}^{baseline} ( f )}^{2} = AVERAGE ( ( {{F}_{k} ( f , t )}^{2} ) ) , t ∈ [ - 200 ms , 0 ] \qquad ( 2 )$$

$$Δ ERSP ( f , t ) = \frac{1}{m} {\sum }_{k = 1}^{m} [ ( {{F}_{k} ( f , t )}^{2} ) - ( {{F}_{k}^{baseline} ( f )}^{2} ) ] \qquad ( 3 )$$

where, Fₖ^{baseline}(f)² represents an average power value in a period of [−200 ms, 0] corresponding to the frequency f in the kth trial; ΔERSP (f, t) represents relative ERSP at the frequency of f and the moment of t after removal of baseline power; and

selecting the relative ERSP at a narrow band over a specific time period by the signal processing module through the following formula,

$$Δ {ERSP}_{ave} = \frac{1}{Δ t} * \frac{1}{Δ f} {\sum }_{f = {f}_{min}}^{{f}_{max}} {\sum }_{t = {t}_{min}}^{{t}_{max}} Δ ERSP ( f , t ) \qquad ( 4 )$$

where, fₘᵢₙ and fₘₐₓ are a lower limit and an upper limit of a narrow band frequency respectively, tₘᵢₙ and tₘₐₓ are a lower limit and an upper limit of interception time respectively, fₘᵢₙ and fₘₐₓ are 38 Hz and 42 Hz in default respectively, and tₘᵢₙ and tₘₐₓ are 1 s and 2.5 s respectively.

Furthermore, the signal processing module extracts the ITC feature, which includes the following steps:

extracting the EEG signals induced by 40 Hz chirp stimulation under montage of the frontal lobes and the temporal lobes through short-time Fourier transform by the signal processing module, and calculating an ITC value according to the following formula:

$$ITC ( f , t ) = \frac{1}{m} {\sum }_{k = 1}^{m} \frac{{F}_{k} ( f , t )}{\vert {F}_{k} ( f , t ) \vert} \qquad ( 5 )$$

where, f and t represent the frequency and time respectively, k is the mark number of the corresponding trial, and m is the total number of n groups of trials;

performing background interference reduction and individual baseline difference on the ASSR data by the signal processing module through the following formula:

ITC^{baseline}(f)=AVERAGE(ITC(f, t)),t∈[200 ms,0] (6)

ΔITC(f, t)=ITC(f, t)−ITC^{baseline}(f) (7)

where, ITC^{baseline} (f) represents an average ITC value in a period of [−200 ms, 0] corresponding to the frequency f; ΔITC(f,t) represents a relative ITC value at the frequency of f and the moment of t after removal of baseline ITC; and

selecting ITC at a narrow band over a specific time period by the signal processing module through the following formula:

$$Δ {ITC}_{ave} = \frac{1}{Δ t} * \frac{1}{Δ f} {\sum }_{f = {f}_{min}}^{{f}_{max}} {\sum }_{t = {t}_{min}}^{{t}_{max}} Δ ITC ( f , t ) \qquad ( 8 )$$

where, fₘᵢₙ and fₘₐₓ are 38 Hz and 42 Hz in default respectively, tₘᵢₙ and tₘₐₓ are 1 s and 2.5 s respectively.

Furthermore, the signal processing module extracts the WPLI feature, which includes the following steps:

measuring WPLI corresponding to a phase angle difference between two time series x(t) and y(t) distributed at positive and negative parts of an imaginary axis in a complex plane by the signal processing module through the following formula:

$$WPLI = \vert \frac{{\sum }_{t = 1}^{n} \vert imag ( {S}_{xy , t} ) \vert sgn ( imag ( {S}_{xy , t} ) )}{{\sum }_{t = 1}^{n} \vert imag ( {S}_{xy , t} ) \vert} \vert \qquad ( 9 )$$

where, S_{xy,t} represents a composite cross spectral density of x(t) and y(t) at the moment of t, and | | represents the evaluation of an absolute value.

Furthermore, the depression detection module identifies the user depression state through decision fusion of the depressive disorder-related EEG features, which includes the following steps:

reducing dimensions of a depressive disorder-related EEG feature matrix by the depression detection module through a sequential backward feature selection algorithm (SBFS) to screen depression classification features;

selecting different depression classification features by the depression detection module by using an SVM classifier as inputs to acquire an identification accuracy rate under each feature;

performing identification and classification on user depression states for the determination of output categories by the depression detection module through decision fusion according to the following formula:

Score^{fusion}=ω₁Score^{ERSP}+ω₂Score^{ITC}+ω₃Score^{WPLI} (10)

where, ω represents a weight coefficient, ω₁, ω₂ and ω₃ are dynamically regulated according to an accuracy rate of a test set during SVM classification with the ERSP, ITC and WPLI as the separate inputs.

The beneficial effects of the present disclosure:

1. The present disclosure enhances stable induction of the gamma ASSR through design of the frequency-increasing chirp signals based on the cochlear traveling wave delay to improve the gamma ASSR signal-to-noise ratio. A mapping relationship between the depressive disorder and the ASSR response is decoded based on a previously constructed electroencephalogram database of normal people versus patients with the depressive disorder, and depressive disorder-related ASSR features are obtained. The comprehensive evaluation of the user depression state is achieved through extraction of multidimensional feature parameters of ERSP, ITC, WPLI representing the functional connectivity from the user ASSR data during auditory stimulation, and the automatic detection and output of the user depression are achieved through decision fusion.

2. The disclosure may achieve early identification of the depressive disorder to assist the user in receiving timely treatment, and may be extended to neuropsychology, clinical medicine and other fields to yield considerable social and economic benefits. In the automatic detection system for the depressive disorder based on the high frequency auditory steady-state response designed by the present disclosure, a frequency-increasing chirp auditory stimulation paradigm capable of stably inducing the gamma ASSR is introduced innovatively, a feasible reference is provided for improvement of ASSR response, and a new method and a new thought are provided for exploration of auditory perception disorders arising from other mental diseases. In addition, an effective approach may be provided for detection of the depressive disorder in conjunction with multidimensional feature parameters of the ASSR induced by the paradigm, and it is expected to provide auxiliary support for precise diagnosis in clinic.

3. The present disclosure is intended to provide the automatic detection system for the depressive disorder based on the high frequency auditory steady-state response, which extracts user ASSR specific feature parameters, namely ERSP, ITC and WPLI, from the acquired EEG data by design of a gamma frequency-increasing chirp auditory stimulation paradigm, and performs accurate and objective automatic detection on the depression through decision fusion. The present disclosure may effectively improve the accuracy and convenience of the automatic detection of the depressive disorder, and yield considerable social and economic benefits.

### BRIEF DESCRIPTION OF THE DRAWINGS

FIG. 1 is a structural schematic diagram of an automatic detection system for depressive disorder based on high frequency auditory steady-state response according to the present disclosure; and

FIG. 2 is a flow chart of a frequency-increasing chirp stimulation paradigm involved in the present disclosure.

### DETAILED DESCRIPTION OF THE EMBODIMENTS

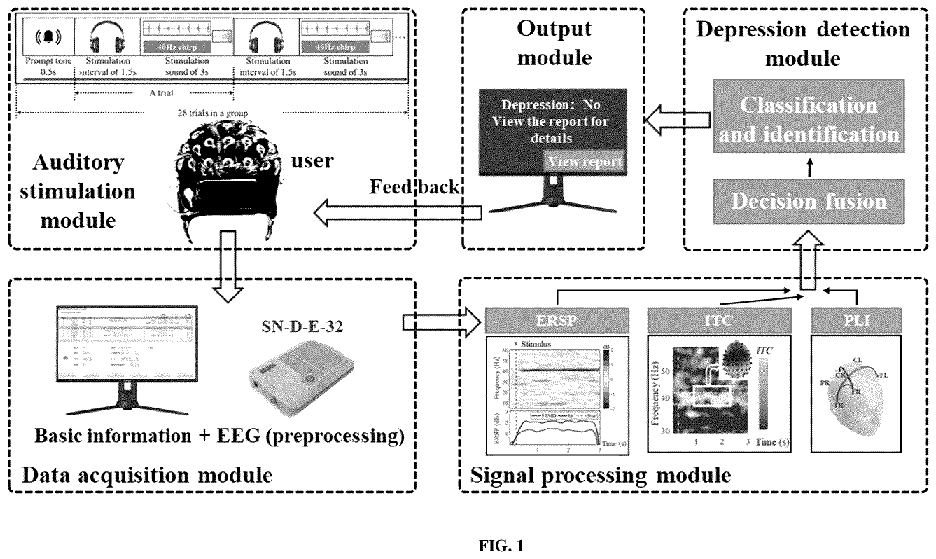

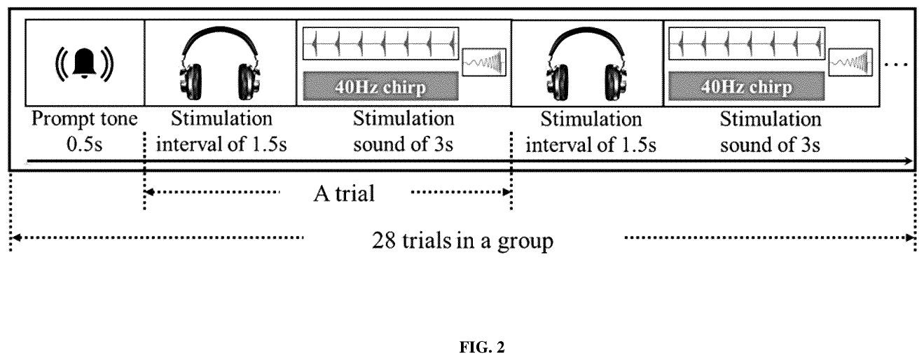

In the present disclosure, the implementation process of the present disclosure will be further described below in detail with reference to FIG. 1 and FIG. 2.

The present disclosure provides an automatic detection system for depressive disorder based on gamma auditory steady-state response (ASSR). The automatic detection system for the depressive disorder in the present disclosure includes an auditory stimulation module, a data acquisition module, a signal processing module, a depression detection module and an output module to achieve automatic detection of a user for depression.

A technical process thereof includes the steps of: designing a frequency-increasing chirp stimulation paradigm capable of effectively inducing gamma ASSR; establishing an electroencephalogram (EEG) information acquisition platform; extracting event-related spectral perturbation (ERSP), inter-trial phase coherence (ITC), a weighted phase-lag-index (WPLI) and other feature parameters which are closely related to the depressive disorder are extracted based on correlation between ASSR response and depressive symptom, found in a preliminary study, classifying and identifying the parameters by a support vector machine (SVM), and finally analyzing the user depression state so as to feed back to the user.

The overall design of the present disclosure is shown in FIG. 1, and the system framework and the technical process include: the auditory stimulation module capable of stably inducing the gamma ASSR is constructed, and the auditory stimulation represented in the module is chirp stimulation designed based on a cochlear traveling wave delay.

During stimulation presentation, scalp EEG signals (namely ASSR) of the user during auditory stimulation are connected to the data acquisition module by an electrode cap, and the data acquisition module applies a developed EEG acquisition system product (digital electroencephalograph, with a model of SN-D-E-32). The acquired scalp EEG signals of the user are transmitted to the signal processing module after being preprocessed by change of reference, filtering and other means, and the signal processing module may extract the feature parameters of ERSP, ITC, WPLI from the user ASSR; then the extracted ASSR feature parameters are input to the depression detection module to achieve automatic identification of the user by using a conventional support vector machine (SVM) and other classification methods based on decision fusion; and the output module feeds detection results to the user more visually.

Functions of each module of the automatic detection system for depressive disorder based on the high frequency auditory steady-state response

### (1) Auditory Stimulation Module

The auditory stimulation module is configured to induce the gamma ASSR of the user. A flow chart of auditory stimulation presentation in the module is shown in FIG. 2. The auditory stimulation is mainly frequency-increasing chirp stimulation. Chirp signals are designed based on the cochlear traveling wave delay, which may enhance synchronous discharge of neurons in the cochlea to improve ASSR response intensity and induce more stable and efficient ASSR. The whole process of the paradigm includes: chirp stimulation for 3 s and interval for 1.5 s are presented in each trial, and the interval time of 1.5 s is set to ensure that no cross impact occurs among stimulations of all trials. The frequency of chirp stimulation is 40 Hz, a sound pressure level is 45 dB, and the sound is played binaurally. 3 groups of sound stimulations are presented in default (the groups may be increased according to clinical needs), and there are a total of 28 trials in each group. The sound is sent to the user by head-mounted wired noise-canceling headphones (model: Edifier W820NB), and the user only needs to keep as still as possible during stimulation, without any other operations.

A control over chirp stimulation is achieved using Matlab2020a (The MathWorks. Inc. Natick, MA) with the Psychtoolbox, including control over a stimulation duration, stimulation intensity and other parameters; and meanwhile, synchronous event codes are sent to an electroencephalograph during stimulation presentation to ensure data synchronization.

### (2) Data Acquisition Module

The data acquisition module is configured to acquire basic information of the user, which includes user age, gender and the like. The module is configured to perform data acquisition and preprocessing for cerebral cortical response (EEG information), induced by the auditory stimulation module, of the user. Wherein, the EEG information acquisition is conducted by a 32-lead digital electroencephalograph (model: SN-D-E-32) self-developed by Datian Medical Engineering (Tianjin) Co., Ltd., which is non-invasive safe and reliable portable EEG acquisition equipment, a data sampling frequency is 1000 Hz, a hardware bandpass filtering ranges from 0.5 Hz to 100 Hz, and a 50 Hz notching filter is adopted for eliminating power frequency interference. The scalp EEG information of the user is connected to the electroencephalograph by a 32-lead electrode cap, and the spatial distribution of electrodes meets an international 10-20 system; a grounding electrode is located at an ‘AFZ’ position of a forehead, a reference electrode is located at an ‘A1’ position of a left mastoid, and the electrode impedance is lower than 10 kΩ. The preprocessing of the EEG information includes change of reference, that is, the left mastoid reference ‘A1’ is changed to average reference [(′A1+A2′)/2] of bilateral mastoids, to reduce influence caused by the lateralization of the reference electrode position. Then, bandpass filtering is conducted across 1 Hz to 50 Hz, the ASSR data is intercepted segmentally according to a time label induced by stimulation, and abnormal trials with an amplitude of no less than 100 μV are eliminated.

### (3) Signal Processing Module

The signal processing module is configured to extract features from the received ASSR data preprocessed by the data acquisition module, and the extracted feature parameters mainly include event-related spectral perturbation (ERSP), inter-trial phase coherence (ITC) and a weighted phase-lag-index (WPLI). Wherein, the ERSP is mainly used for measuring 40 Hz entrainment response power variation, the ITC is mainly used for measuring an ITC value, and the WPLI is mainly used for measuring a change in the connectivity between the brain regions during 40 Hz chirp stimulation.

### (3.1) ERSP Feature Extraction

In the present disclosure, the frequency domain feature is analyzed by short-time Fourier transform (STFT). The ASSR signals induced by 40 Hz chirp stimulation under montage of the frontal lobes and the temporal lobes are extracted, and a power value of each trial is calculated according to the following formula:

$$ERSP ( f , t ) = \frac{1}{m} {\sum }_{k = 1}^{m} ( {{F}_{k} ( f , t )}^{2} ) \qquad ( 1 )$$

where, f represents frequency, t represents time, k is a mark number of a corresponding trial, m is a total number of n groups of trials (28 trials/group*3 in default), and Fₖ(f,t)² represents a corresponding power value at a frequency of f and the moment of t in a kth trial.

In order to reduce background interference and individual baseline difference, baseline removal processing is further conducted according to the following formula:

$${{F}_{k}^{baseline} ( f )}^{2} = AVERAGE ( ( {{F}_{k} ( f , t )}^{2} ) ) , t ∈ [ - 200 ms , 0 ] \qquad ( 2 )$$

$$Δ ERSP ( f , t ) = \frac{1}{m} {\sum }_{k = 1}^{m} [ ( {{F}_{k} ( f , t )}^{2} ) - ( {{F}_{k}^{baseline} ( f )}^{2} ) ] \qquad ( 3 )$$

Where, Fₖ^{baseline} (f)² represents an average power value in a period [−200 ms, 0] corresponding to the frequency f in the kth trial (the baseline time is selected from the beginning of the stimulation, that is the moment 0, to −200 ms before stimulation in default).

ΔERSP(f, t) represents the relative ERSP at the frequency of f and the moment of t after removal of the baseline power. The relative ERSP at a narrow band over a specific time period is selected according to the clinical needs, and individual average relative 40 Hz ASSR ERSP is obtained by a stacking average method according to the following formula:

$$Δ {ERSP}_{ave} = \frac{1}{Δ t} * \frac{1}{Δ f} {\sum }_{f = {f}_{min}}^{{f}_{max}} {\sum }_{t = {t}_{min}}^{{t}_{max}} Δ ERSP ( f , t ) \qquad ( 4 )$$

where, fₘᵢₙ and fₘₐₓ are a lower limit and an upper limit of a narrow band frequency respectively, tₘᵢₙ and tₘₐₓ are a lower limit and an upper limit of interception time respectively, fₘᵢₙ and fₘₐₓ are 38 Hz and 42 Hz in default respectively, and train and tₘₐₓ are 1 s and 2.5 s respectively.

### (3.2) ITC Feature Extraction

In the same way, the ASSR signals induced by 40 Hz chirp stimulation under montage of the frontal lobes and the temporal lobes are extracted by the short-time Fourier transform (STFT), and the ITC value is calculated according to the following formula:

$$ITC ( f , t ) = \frac{1}{m} {\sum }_{k = 1}^{m} \frac{{F}_{k} ( f , t )}{\vert {F}_{k} ( f , t ) \vert} \qquad ( 5 )$$

It is similar to the ERSP formula, where, f and t represent the frequency and time respectively, k is the mark number of the corresponding trial, and m is the total number of n groups of trials. Similarly, in order to reduce background interference, the baseline removal processing is further conducted according to the following formula:

ITC^{baseline}(f)=AVERAGE(ITC(f, t)),t∈[−200 ms,0] (6)

ΔITC(f, t)=ITC(f, t)−ITC^{baseline}(f) (7)

where, ITC^{baseline}(f) represents an average ITC value Ma period [−200 ms, 0] corresponding to the frequency f (the baseline is selected from the beginning of the stimulation, that is the moment 0, to −200 ms before stimulation in default). ΔITC(f, t) represents a relative ITC value at the frequency of f and the moment of t after removal of baseline ITC. The ITC at a narrow band over a specific time period is selected, and individual average relative 40 Hz ASSR ITC is obtained by the stacking average method according to the following formula:

$$Δ {ITC}_{ave} = \frac{1}{Δ t} * \frac{1}{Δ f} {\sum }_{f = {f}_{min}}^{{f}_{max}} {\sum }_{t = {t}_{min}}^{{t}_{max}} Δ ITC ( f , t ) \qquad ( 8 )$$

where, fₘᵢₙ and fₘₐₓ are 38 Hz and 42 Hz in default respectively, tₘᵢₙ and tₘₐₓ are 1 s and 2.5 s respectively.

### (3.3) WPLI Feature Extraction

WPLI is configured to measure a degree of a phase angle difference between two time series x(t) and y(t) distributed at positive and negative parts of an imaginary axis in a complex plane according to the following formula:

$$WPLI = \vert \frac{{\sum }_{t = 1}^{n} \vert imag ( {S}_{xy , t} ) \vert sgn ( imag ( {S}_{xy , t} ) )}{{\sum }_{t = 1}^{n} \vert imag ( {S}_{xy , t} ) \vert} \vert \qquad ( 9 )$$

where, S_{xy,t} represents a composite cross spectral density of x(t) and y(t) at the moment of t, and | | represents the evaluation of an absolute value. In the present disclosure, the average WPLI in a time interval of 1 s to 2.5 s within a narrow band ranging from 38 Hz to 42 Hz is calculated, and 8 brain regions are divided, including right and left frontal lobes, right and left temporal lobes, right and left central regions, and right and left parietal lobes; and the connectivity between every two brain regions is calculated through montage averaging, and finally is subjected to block averaging at a trial level.

### (4) Depression Detection Module

The depression detection module is configured to detect and evaluate whether the user gets depressed by using the SVM and the decision fusion according to the ASSR features (ERSP, ITC and WPLI) transmitted from the signal processing module. It is specifically shown as follows:

The dimensions of (ERSP, ITC and WPLI) feature matrices are reduced by using a sequential backward feature selection algorithm (SBFS), and preferred depression classification features are screened out; and then, different depression classification features are selected by using the SVM classifier as inputs to obtain the identification accuracy rate under each feature.

Finally, identification and classification are conducted through decision fusion, that is, for the fusion method of classification results of ERSP, ITC and WPLI of 40 Hz ASSR, linear weighted fusion is adopted to determine a final output category, as shown in the following formula:

Score^{fusion}=ω₁Score^{ERSP}+ω₂Score^{ITC}+ω₃Score^{WPLI} (10)

where, ω represents a weight coefficient, ω₁, ω₂ and ω₃ are dynamically regulated according to an accuracy rate of a test set during SVM classification with the ERSP, ITC and WPLI features as the separate inputs. For example, when the ERSP feature is input separately, the classification accuracy rate of a test set is a %; when the ITC feature is input separately, the classification accuracy rate of the test set is b %; when the WPLI feature is input separately, the classification accuracy rate of a test set is c %, under which the weight coefficient ω₁ is a/(a+b+c), ω₂ is b/(a+b+c), and ω₃ is c/(a+b+c).

### (5) Output Module

A final detection result and an evaluation report are fed back to the user by a liquid crystal display connected to the detection module, and the user may save or print the detection result.

The present disclosure is not limited to the implementations as described above. The above description of the specific implementations aims to describe and explain the technical solutions of the present disclosure, and the above specific implementations are only exemplary instead of restrictive. Those ordinarily skilled in the art may make concrete transformations in various forms without departing from the purpose of the present disclosure and the scope protected by the claims under inspiration of the present disclosure, and these transformations all belong to the protection scope of the present disclosure.

What is claimed is:

1. An automatic detection system for depressive disorder based on high frequency auditory steady-state response (ASSR), comprising an auditory stimulation module, a data acquisition module, a signal processing module, a depression detection module and an output module, wherein
the auditory stimulation module being configured to input 40 Hz frequency-increasing sound stimulation signals to a user to obtain electroencephalogram (EEG) signals;
the data acquisition module being configured to obtain ASSR data by acquiring the EEG signals by a non-intrusive method and performing preprocessing to the signal; the signal processing module being configured to extract depressive disorder-related EEG features from the ASSR data; wherein
the depressive disorder-related EEG features comprising an event-related spectral perturbation (ERSP) feature, an inter-trial phase coherence (ITC) feature and a weighted phase-lag-index (WPLI) feature;
the ERSP feature being configured to measure power change in 40 Hz ASSR;
the ITC feature being configured to measure an ITC value during auditory stimulation;
the WPLI feature representing the connectivity between the brain regions being configured to measure a change in the connectivity between the brain regions during auditory stimulation;
the depression detection module being configured to identify a user depression state through decision fusion of the depressive disorder-related EEG features; and
the output module being configured to identify the user depression state according to the EEG features to generate an evaluation report for abnormality in EEG response, and feed it back to the user.

2. The automatic detection system for the depressive disorder based on the high frequency ASSR according to claim 1, wherein
extracting the ERSP feature by the signal processing module comprises the following steps:
performing, by the signal processing module, on frequency domain feature analysis through short-time Fourier transform, extracting the ASSR data induced by 40 Hz frequency-increasing stimulation under montage of frontal lobes and temporal lobes, and calculating a power value of each trial according to the following formula:
$$ERSP ( f , t ) = \frac{1}{m} {\sum }_{k = 1}^{m} ( {{F}_{k} ( f , t )}^{2} ) \qquad ( 1 )$$
where, f represents frequency, t represents time, k is a mark number of a corresponding trial, m is a total number of n groups of trials, Fₖ (f, t)² represents a corresponding power value at a frequency of f and the moment of t in a kth trial;
performing, by the signal processing module, background interference reduction and individual baseline difference on the ASSR data through the following formula,
$${{F}_{k}^{baseline} ( f )}^{2} = AVERAGE ( ( {{F}_{k} ( f , t )}^{2} ) ) , t ∈ [ - 200 ms , 0 ] \qquad ( 2 )$$
$$Δ ERSP ( f , t ) = \frac{1}{m} {\sum }_{k = 1}^{m} [ ( {{F}_{k} ( f , t )}^{2} ) - ( {{F}_{k}^{baseline} ( f )}^{2} ) ] \qquad ( 3 )$$
where, Fₖ^{baseline}(f)² represents an average power value in a period of [−200 ms, 0] corresponding to the frequency f in the kth trial; ΔERSP(f, t) represents relative ERSP at the frequency off and the moment of t after removal of baseline power; and
selecting, by the signal processing module, the relative ERSP at a narrow band over a specific time period through the following formula:
$$Δ {ERSP}_{ave} = \frac{1}{Δ t} * \frac{1}{Δ f} {\sum }_{f = {f}_{min}}^{{f}_{max}} {\sum }_{t = {t}_{min}}^{{t}_{max}} Δ ERSP ( f , t ) \qquad ( 4 )$$
where, fₘᵢₙ and fₘₐₓ are a lower limit and an upper limit of a narrow band frequency respectively, and tₘᵢₙ and tₘₐₓ are a lower limit and an upper limit of interception time respectively, and fₘᵢₙ and fₘₐₓ are 38 Hz and 42 Hz in default respectively, and tₘᵢₙ and tₘₐₓ are 1 s and 2.5 s respectively.

3. The automatic detection system for the depressive disorder based on the high frequency ASSR according to claim 1, wherein
extracting the ITC feature by the signal processing module comprises the following steps:
extracting, by the signal processing module, EEG signals induced by 40 Hz frequency-increasing stimulation under montage of the frontal lobes and the temporal lobes through short-time Fourier transform (STFT), and calculating an ITC value according to the following formula:
$$ITC ( f , t ) = \frac{1}{m} {\sum }_{k = 1}^{m} \frac{{F}_{k} ( f , t )}{\vert {F}_{k} ( f , t ) \vert} \qquad ( 5 )$$
where, f and t represent the frequency and time respectively, k is the mark number of the corresponding trial, and m is the total number of n groups of trials;
performing, by the signal processing module, reduction of background interference and individual baseline difference on the ASSR data through the following formula:
ITC^{baseline}(f)=AVERAGE(ITC(f, t)),t∈[−200 ms, 0] (6)
ΔITC(f, t)=ITC(f, t)−ITC^{baseline}(f) (7)
where, ITC^{baseline}(f) represents an average ITC value in a period of [−200 ms, 0] corresponding to the frequency f; ΔITC(f,t) represents a relative ITC value at the frequency of f and the moment of t after removal of baseline ITC; and
selecting, by the signal processing module, the ITC at a narrow band over a specific time period through the following formula
$$Δ {ITC}_{ave} = \frac{1}{Δ t} * \frac{1}{Δ f} {\sum }_{f = {f}_{min}}^{{f}_{max}} {\sum }_{t = {t}_{min}}^{{t}_{max}} Δ ITC ( f , t ) \qquad ( 8 )$$
where, fₘᵢₙ and fₘₐₓ are 38 Hz and 42 Hz in default respectively, and train and tₘₐₓ are 1 s and 2.5 s respectively.

4. The automatic detection system for the depressive disorder based on the high frequency ASSR according to claim 1, wherein
extracting, by the signal processing module, the WPLI feature comprises the following steps:
measuring, by the signal processing module, WPLI corresponding to a phase angle difference between two time series x(t) and y(t) distributed at positive and negative parts of an imaginary axis in a complex plane through the following formula; and
$$WPLI = \vert \frac{{\sum }_{t = 1}^{n} \vert imag ( {S}_{xy , t} ) \vert sgn ( imag ( {S}_{xy , t} ) )}{{\sum }_{t = 1}^{n} \vert imag ( {S}_{xy , t} ) \vert} \vert \qquad ( 9 )$$
where, S_{xy,t} represents a composite cross spectral density of x(t) and y(t) at the moment of t, and | | represents the evaluation of an absolute value.

5. The automatic detection system for the depressive disorder based on the high frequency ASSR according to claim 1, wherein
identifying, by the depression detection module, the user depression state through decision fusion of the depressive disorder-related EEG features comprises the following steps:
reducing, by the depression detection module, dimensions of a depressive disorder-related EEG feature matrix through a sequential backward feature selection algorithm (SBFS) to screen depression classification features;
selecting, by the depression detection module, different depression classification features using a support vector machine (SVM) classifier as inputs to acquire an identification accuracy rate under each feature;
performing, by the depression detection module, identification and classification on user depression states for the determination of output categories through decision fusion according to the following formula; and
Score^{fusion}=ω₁Score^{ERSP}+ω₂Score^{ITC}+ω₃Score^{WPLI} (10)
where, ω represents a weight coefficient ω₁, ω₂ and ω₃ are dynamically regulated according to an accuracy rate of a test set during SVM classification with the ERSP, ITC and WPLI features as the separate inputs.
